(19) United States
(12) Patent Application Publication
(10) Pub. No.: US 2026/0207833 A1
(43) Pub. Date: Jul. 23, 2026

(54) Oscillating ECMO Oxygenator

(71) Applicant: Breethe, Inc., Halethorpe, MD (US)

(72) Inventor: Ryan Orizondo, Danvers, MA (US)

(73) Assignee: Breethe, Inc., Halethorpe, MD (US)

(21) Appl. No.: 19/457,671

(22) Filed: Jan. 23, 2026

Related U.S. Application Data
(60) Provisional application No. 63/748,482, filed on Jan. 23, 2025.

(51) Int. Cl. A61M 1/16; A61M 1/26; B01D 19/00; B01D 63/02; B01D 63/16

(52) U.S. Cl. CPC A61M 1/1698; B01D 19/0031; B01D 63/02; B01D 63/16; A61M 1/26; A61M 2205/10; B01D 2313/201

(57) ABSTRACT

An extracorporeal membrane oxygenation (ECMO) system for infusing blood with oxygen and for removing carbon dioxide from blood includes a blood oxygenator. The blood oxygenator includes a housing having a first end and a second end opposite the first end, and a fiber bundle disposed within the housing and in contact with the first and second ends of the housing. While blood is in contact with the fiber bundle, the first and second ends of the housing are configured to move towards one another and create oscillatory motion of the fibers within the bundle to enhance gas transfer and surface washing within the bundle.

### CROSS-REFERENCE TO RELATED APPLICATIONS

This application claims the benefit of the filing date of U.S. Provisional Ser. No. 63/748,482 , filed Jan. 23, 2025, the disclosure of which is hereby incorporated herein by reference.

### BACKGROUND OF THE INVENTION

The present disclosure relates to the recovery of a patient undergoing extracorporeal membrane oxygenation (ECMO), sometimes also referred to as extracorporeal life support (ECLS), and more particularly to an oxygenator used to oxygenate a patient's blood during ECMO therapy.

Patients awaiting or recovering from a heart or lung transplant and patients having conditions putting them at risk for heart or lung failure may be candidates for ECMO therapy. ECMO therapy is designed to address an excess of carbon dioxide and/or lack of oxygen in the blood where the lungs are not healthy and/or the heart cannot pump enough blood around the body. ECMO therapy is thus an extracorporeal technique which replaces these respiratory and cardiac functions in various situations ranging from support needed while separately treating the underlying causes of cardiac arrest to late-stage treatment for heart or lung failure. It may be a therapeutic treatment to provide temporary help when needed.

ECMO therapy operates by drawing deoxygenated blood from the body of a patient in a controlled manner to permit oxygenation of the blood and to remove carbon dioxide from the blood. The oxygenated blood is then cycled back into the patient. Generally, ECMO systems include a pump, an oxygenator, an oxygen source and a control center to monitor and control the process. It may also include a blood warmer to bring the blood back up to the temperature of the body as it is circulated back into the body. Deoxygenated blood drawn from the body via a cannula is pumped through an oxygenator where oxygen is infused in the blood and carbon dioxide is removed. The oxygenated blood is pumped from the oxygenator and back into the body, with blood warming in most instances.

### BRIEF SUMMARY OF THE INVENTION

In accordance with an embodiment of a first aspect of the present disclosure, a blood oxygenator includes a housing having a first end and a second end opposite the first end, a fiber bundle disposed within the housing and in contact with the first and second ends of the housing, wherein the first and second ends of the housing are configured to move towards one another and create oscillatory motion of the fibers within the bundle.

In another embodiment of the first aspect, a first end of the fiber bundle is in contact with the first end of the housing and a second end of the fiber bundle is in contact with the second end of the housing. In yet another embodiment of the first aspect, the first and second ends of the fiber bundle comprise potted layers of fibers. In yet another embodiment of the first aspect, the first and second ends of the housing are configured to move towards one another via compression. In yet another embodiment of the first aspect, the first and second ends of the housing are configured to move relative to one another via torsion. In yet another embodiment of the first aspect, the first and second ends of the housing are configured to move towards one another by a distance less than 1 centimeter.

In yet another embodiment of the first aspect, the housing has a first portion extending to the first end of the housing and a second portion extending to the second end of the housing. In yet another embodiment of the first aspect, a seam is connecting the first portion and the second portion of the housing. In yet another embodiment of the first aspect, the seam comprises a compressible material. In yet another embodiment of the first aspect, the seam comprises a gasket.

In accordance with an embodiment of a second aspect of the present disclosure, a blood oxygenator includes a housing having an oxygenator fiber bundle, a blood inlet to the fiber bundle, a blood outlet, an oxygen inlet to the fiber bundle for oxygenating the blood of a patient and an oxygen outlet; an oxygen source to provide oxygen to the oxygen inlet; and a pump for pumping oxygen through an oxygen feed conduit to the fiber bundle, wherein the housing is configured to move the fiber bundle while contacting the blood of the patient.

In another embodiment of the second aspect, the housing is configured to oscillate the fiber bundle. In yet another embodiment of the second aspect, a first end of the fiber bundle is in contact with a first end of the housing and a second end of the fiber bundle is in contact with a second end of the housing. In yet another embodiment of the second aspect, the first and second ends of the fiber bundle comprise potted layers of fibers. In yet another embodiment of the second aspect, the first and second ends of the housing are configured to move towards one another via compression. In yet another embodiment of the second aspect, the first and second ends of the housing are configured to move relative to one another via torsion. In yet another embodiment of the second aspect, the first and second ends of the housing are configured to move towards one another by a distance less than 1 centimeter.

In yet another embodiment of the second aspect, the housing has a first portion extending to the first end of the housing and a second portion extending to the second end of the housing. In yet another embodiment of the second aspect, a seam is connecting the first portion and the second portion of the housing. In yet another embodiment of the second aspect, the seam comprises a compressible material. In yet another embodiment of the second aspect, the seam comprises a gasket.

In accordance with an embodiment of a third aspect of the present disclosure, a method of oxygenating blood of a patient includes providing a housing having within it a fiber bundle, a blood inlet, a blood outlet, an oxygen inlet and an oxygen outlet; directing blood from a patient through the blood inlet into the fiber bundle; pumping oxygen through an oxygen feed conduit extending from an oxygen source to the oxygen inlet; and moving a portion of the fiber bundle to infuse the blood with oxygen.

In another embodiment of the third aspect, the step of moving a portion of the fiber bundle comprises compressing the fiber bundle with the housing. In yet another embodiment of the third aspect, the step of moving a portion of the fiber bundle comprises inducing a torsional translation of the fibers within the bundle with the housing. In yet another embodiment of the third aspect, the step of moving a portion of the fiber bundle comprises oscillating fibers of the fiber bundle to create a bowing motion.

### BRIEF DESCRIPTION OF THE DRAWINGS

FIG. 1 is a schematic representation of an ECMO system in accordance with an embodiment of the present disclosure;

FIG. 2 is a perspective view of a blood oxygenator of the EMCO system of FIG. 1;

FIG. 3 is a cross-sectional view of the blood oxygenator of FIG. 2;

FIG. 4 is a perspective view of a fiber bundle of the blood oxygenator of FIG. 2; and

FIG. 5 is another cross-sectional view of the blood oxygenator of FIG. 2.

### DETAILED DESCRIPTION

Oxygenators may be used to oxygenate a patient's blood during ECMO therapy. Oxygenators currently use bundles of semipermeable, hollow fibers that are designed for facilitating gas transfer of oxygen and carbon dioxide when contacting blood. These fiber bundles are typically stationary within the oxygenator and are comprised of layers of adjacent fiber mats, which create crossing points at locations where the fiber mats contact one another. After extended use of these currently available oxygenators, concentrations of biodeposits from patients'blood may build up at these crossing points of the fiber mats and may create a nidus for biological material formation. Biological material formation may cause a reduction in oxygenator performance, which may be critical for patients during ECMO therapy. As such, it would be desirable for an improved oxygenator to induce movement of these fibers relative to each other to transiently change the points of contact between adjacent fibers, thereby allowing for enhanced local surface washing at these crossing points and to mitigate biodeposit buildup.

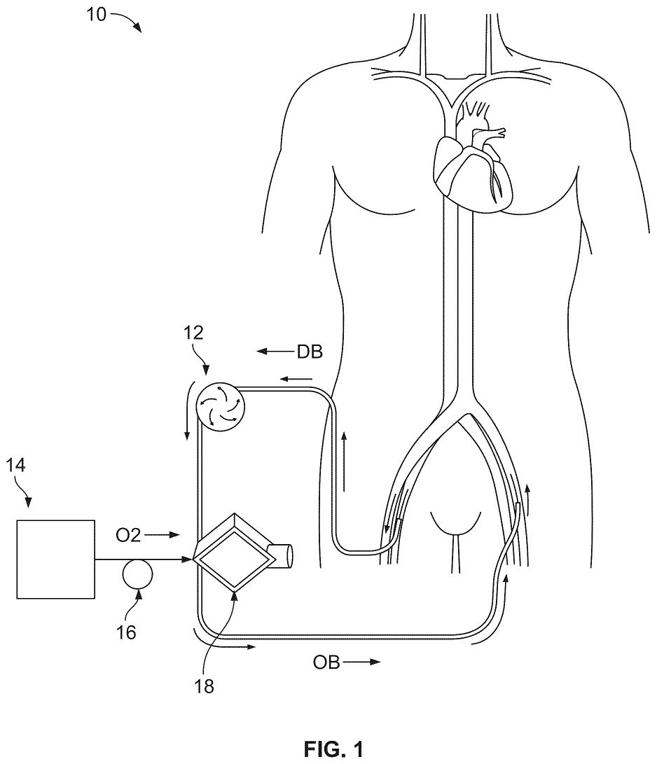

Referring to FIG. 1, an ECMO system 10 is shown which includes a blood pump 12, an oxygen source 14, an oxygen pump or other pressurized gas source 16 and a blood oxygenator 18.

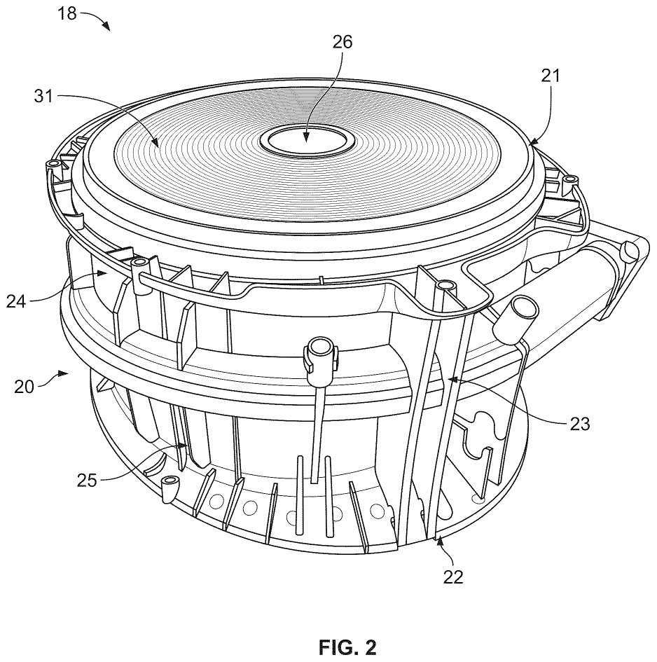

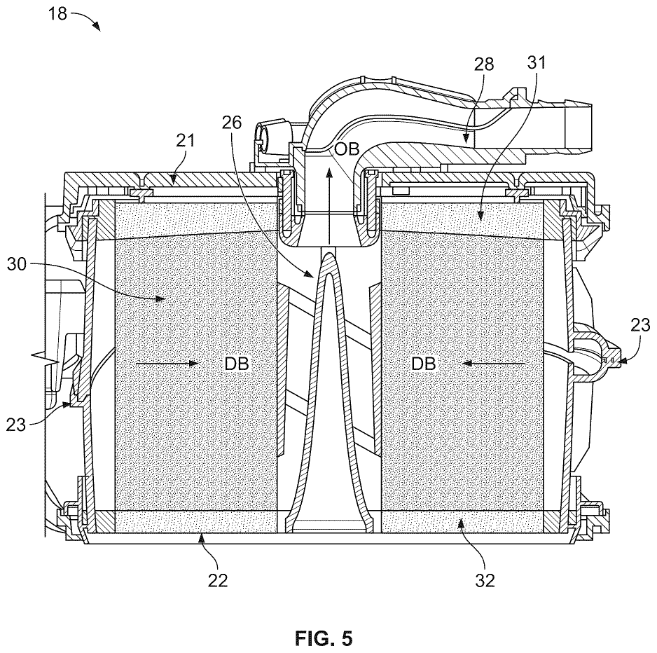

Shown more specifically in FIGS. 2-5, oxygenator 18 includes a housing 20 and a bundle of hollow fibers 30. Housing 20 extends from a first end 21 to a second end 22 opposite first end 21. A seam or gasket 23 circumferentially extends along an outer surface of housing 20. Seam 23 connects a first portion 24 of housing 20 extending to first end 21, to a second portion 25 of housing extending to second end 22. A conduit 26 extends through a center of the housing from first end 21 to the interior of oxygenator 18 and defines an outlet 28 for oxygenated blood OB to exit oxygenator 18 and be recirculated within the patient, as discussed herein.

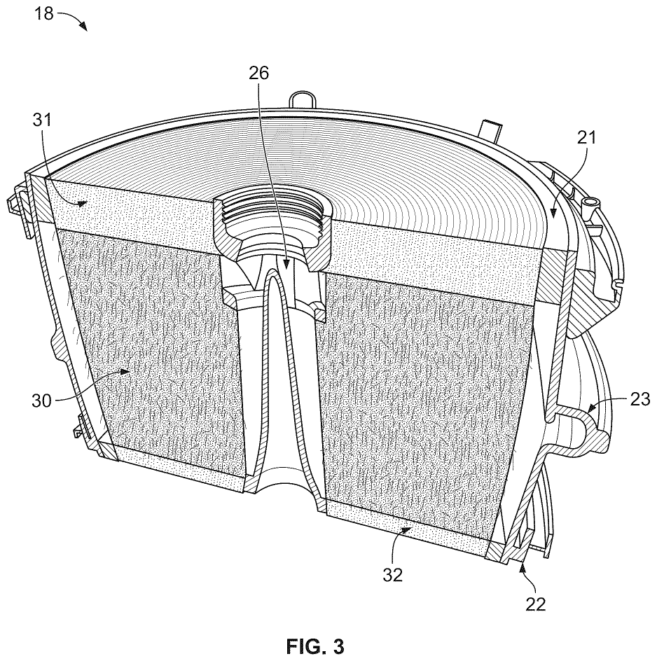

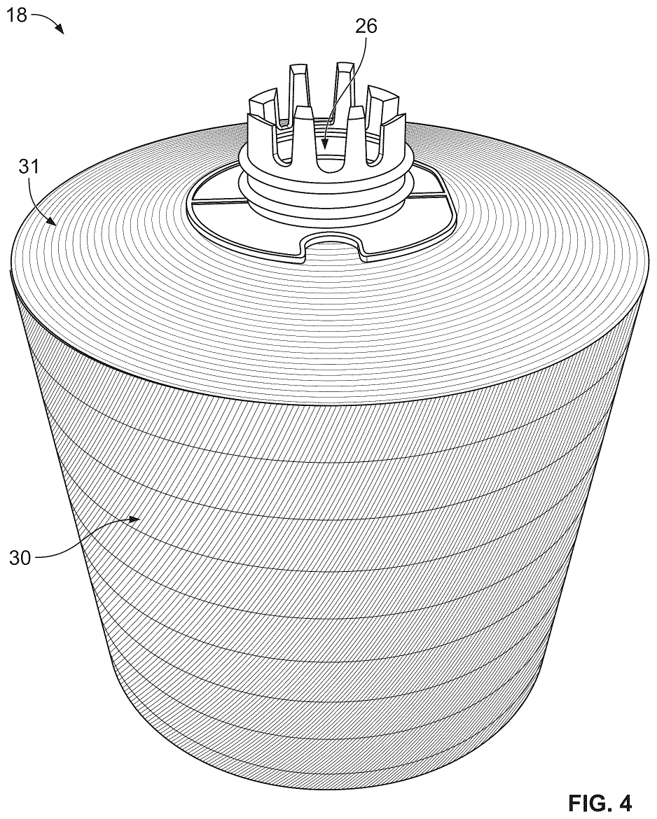

Fiber bundle 30 is made of semi-permeable hollow fibers, as best shown in FIGS. 3 and 4, and a first end 31 and a second end 32 of fiber bundle 30 are potted. Fiber bundle 30 is positioned within housing 20 such that first potted end 31 is adjacent to first end 21 of housing 20 and second potted end 32 is adjacent to second end 22 of housing 20. Fiber bundle 30 circumferentially surrounds conduit 26 when positioned within housing 20. Fiber bundle 30 may comprise a polymethylpentene (PMP) hollow fiber membrane mesh, in the form of a straw tube-like structures, with each fiber comprising a gas permeable wall. Further, in some examples, fiber bundle 30 may comprise approximately 30,000 fibers (each 380 microns in diameter) woven into an array, restraining and aligning the fibers in an axial direction and wound into a annular shape.

In use, deoxygenated blood DB is pumped into oxygenator 18 and passes through fiber bundle 30 in a radial direction until reaching conduit 26. As deoxygenated blood DB is pumped through fiber bundle 30, gas flows through the lumens of each of the fibers which causes oxygen and carbon dioxide diffusion into and out of the blood, respectively. After reaching conduit 26, oxygenated blood OB exits oxygenator 18 through outlet 28 to be recirculated within the patient.

As deoxygenated blood DB passes through fiber bundle 30, oxygenator 18 induces oscillatory movement or motion of the fibers. First and second ends 21, 22 of housing 20 are configured to move first and second potted ends 31, 32 of fiber bundle 30 relative to and towards one another within housing 20 and create a bowing motion of the fibers. The induced movement of first and second potted ends 31, 32 of fiber bundle 30 may be caused by compression or vertical translations of each respective ends relative to one another, and such compression may be created from mechanical, pneumatic or piezoelectric means. Alternatively, the induced movement of the first and second potted ends 31, 32 of fiber bundle 30 may be caused by rotational or torsional translations of each respective end relative to one another, and such torsion may be created from mechanical, pneumatic or piezoelectric means. As first and second ends 21, 22 of housing 20 move towards one another, seam 23, comprised of flexible material or gasket, compresses to allow the induced movement of first and second potted ends 31, 32 of fiber bundle 30. Further, in some examples, a distance of the induced movement of each of the first and second potted ends 31, 32 from a stationary position may be less than 1 centimeter. In alternative embodiments, the first end 21 of housing 20 moves towards the second end 22 of housing 20 while second end 22 of housing remains stationary, or vice versa.

Oscillatory motion of fibers within fiber bundle 30 reduces oxygenator 18 thrombogenicity and improves oxygenator 18 gas transfer efficiency. Induced oscillatory motion of the fibers increases efficiency of fiber bundle 30 and reduces thrombogenicity which will extend the useable lifetime of oxygenator 18. Additionally, improving gas transfer efficiency of the fibers will enable increased overall gas transfer capacity (i.e., support higher blood flow rates) and reduce overall fiber surface area without sacrificing capacity. Further, a reduction in the overall fiber surface area would enable a more compact oxygenator 18 and ECMO system 10 for enhanced patient ambulation.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A blood oxygenator, comprising:
a housing having a first end and a second end opposite the first end;
a fiber bundle disposed within the housing and in contact with the first and second ends of the housing,
wherein the first and second ends of the housing are configured to move towards one another and oscillate the fiber bundle.

2. The blood oxygenator of claim 1, wherein a first end of the fiber bundle is in contact with the first end of the housing and a second end of the fiber bundle is in contact with the second end of the housing.

3. The blood oxygenator of claim 2, wherein the first and second ends of the fiber bundle comprise potted layers of fibers.

4. The blood oxygenator of claim 1, wherein the first and second ends of the housing are configured to move towards one another via compression.

5. The blood oxygenator of claim 1, wherein the first and second ends of the housing are configured to move relative to one another via torsion.

6. The blood oxygenator of claim 1, wherein the first and second ends of the housing are configured to move towards one another by a distance less than 1 centimeter.

7. The blood oxygenator of claim 1, wherein the housing has a first portion extending to the first end of the housing and a second portion extending to the second end of the housing.

8. The blood oxygenator of claim 7, further comprising a seam connecting the first portion and the second portion of the housing.

9. A blood oxygenator, comprising:
a housing having an oxygenator fiber bundle, a blood inlet to the fiber bundle, a blood outlet, an oxygen inlet to the fiber bundle for oxygenating the blood of a patient and an oxygen outlet;
an oxygen source to provide oxygen to the oxygen inlet; and
a pump for pumping oxygen through an oxygen feed conduit to the fiber bundle, wherein the housing is configured to move the fiber bundle while contacting the blood of the patient.

10. The blood oxygenator of claim 9, wherein the housing is configured to oscillate the fiber bundle.

11. The blood oxygenator of claim 9, wherein a first end of the fiber bundle is in contact with a first end of the housing and a second end of the fiber bundle is in contact with a second end of the housing.

12. The blood oxygenator of claim 11, wherein the first and second ends of the fiber bundle comprise potted layers of fibers.

13. The blood oxygenator of claim 11, wherein the first and second ends of the housing are configured to move towards one another via compression.

14. The blood oxygenator of claim 11, wherein the first and second ends of the housing are configured to move relative to one another via torsion.

15. The blood oxygenator of claim 11, wherein the first and second ends of the housing are configured to move towards one another by a distance less than 1 centimeter.

16. The blood oxygenator of claim 11, wherein the housing has a first portion extending to the first end of the housing and a second portion extending to the second end of the housing.

17. A method of oxygenating blood of a patient, comprising:
providing a housing having within it a fiber bundle, a blood inlet, a blood outlet, an oxygen inlet and an oxygen outlet;
directing blood from a patient through the blood inlet into the fiber bundle;
pumping oxygen through an oxygen feed conduit extending from an oxygen source to the oxygen inlet; and
moving a portion of the fiber bundle to infuse the blood with oxygen.

18. The method of claim 17, wherein the step of moving a portion of the fiber bundle comprises compressing the fiber bundle with the housing.

19. The method of claim 17, wherein the step of moving a portion of the fiber bundle comprises inducing a torsional translation of the fiber bundle with the housing.

20. The method of claim 17, wherein the step of moving a portion of the fiber bundle comprises oscillating fibers of the fiber bundle to create a bowing motion.
